(19) United States
(12) Patent Application Publication
(10) Pub. No.: US 2020/0072822 A1
(43) Pub. Date: Mar. 5, 2020

(54) EXOSOME ANALYSIS METHOD, EXOSOME ANALYSIS CHIP, AND EXOSOME ANALYSIS DEVICE

(71) Applicants: THE UNIVERSITY OF TOKYO, Tokyo (JP); NIKON CORPORATION, Tokyo (JP)

(72) Inventors: Takanori ICHIKI, Tokyo (JP); Takanori AKAGI, Tokyo (JP); Kuno SUZUKI, Iruma-gun (JP)

(21) Appl. No.: 16/679,831

(22) Filed: Nov. 11, 2019

Related U.S. Application Data
(62) Division of application No. 15/053,345, filed on Feb. 25, 2016.
Application No. 15/053,345 is a continuation of application No. PCT/JP2014/072252, filed on Aug. 26, 2014.

(30) Foreign Application Priority Data
Aug. 30, 2013 (JP) 2013-180575

(51) Int. Cl. G01N 33/53; B01L 3/00; G01N 33/543

(52) U.S. Cl. CPC G01N 33/5308; B01L 2300/16; G01N 33/54366; B01L 3/502761; B01L 2400/06; B01L 2300/165; B01L 2300/161; B01L 2300/0627; B01L 2300/0816; B01L 2200/16; B01L 2300/0867; B01L 2200/0647; B01L 2300/0864; B01L 2200/04; B01L 2300/0636; B01L 3/502

(57) ABSTRACT

The exosome analysis method of the present invention comprises (a) bringing an exosome-containing sample into contact with a substrate that is modified with a compound having a hydrophobic chain and a hydrophilic chain to bind the exosome to the compound; (b) bringing the exosome into contact with a first molecule that specifically binds to a biomolecule existing on the surface of the exosome to form a first molecule-exosome complex on the substrate; and (c) detecting the first molecule-exosome complex on the substrate.

### CROSS-REFERENCE TO RELATED APPLICATION

This application is a divisional of U.S. application Ser. No. 15/053,345, filed on Feb. 25, 2016, which is a continuation of International Application No. PCT/JP2014/072252, filed Aug. 26, 2014, which claims priority to Japanese Application No. 2013-180575, filed Aug. 30, 2013, all of which are incorporated verbatim herein by reference in their entirety, including the specifications, drawings, and the claims.

### BACKGROUND

The present invention relates to an exosome analysis method, an exosome analysis chip, and an exosome analysis device.

An exosome is a small lipid vesicle with a diameter of 30 nm to 100 nm, and is secreted in a body fluid such as blood, urine, and saliva from various cells such as tumor cells, dendritic cells, T cells, and B cells, as a fused body of an endosome and a cell membrane.

In some cases, abnormal cells such as cancer cells express a specific protein in a cell membrane. Proteins derived from a cell of a secretion source are expressed on the surface of the membrane of an exosome. Therefore, a technology is expected to be established in which it is possible to examine an abnormality within a living body by analyzing proteins existing on the surface of the membrane of the exosome in a body fluid, even without performing a biopsy examination.

The term “biopsy examination” refers to a clinical examination for making a diagnosis of a disease or the like through observation of a lesion site using a microscope after collecting tissues from the lesion site.

A method for analyzing an exosome using an enzyme-linked immunosorbent assay (ELISA) has been proposed with respect to such an expectation (JP2011-510309A). Specific examples of the ELISA technique include a sandwich method and a direct adsorption method.

The sandwich method is a method for measuring the amount of a signal derived from an exosome contained in a sample as follows. After immobilizing an antibody (hereinafter, referred to as a first antibody) to a protein (hereinafter, referred to as a first protein) which is expressed on the surface of a membrane of an exosome on a solid phase, a complex is formed by bringing a sample containing an exosome into contact with the solid phase; a labeled antibody made by modifying an antibody (hereinafter, referred to as a second antibody) to another protein (hereinafter, referred to as a second protein) which is expressed on the surface of the membrane of the exosome is added to the complex to form a further complex; and the label is detected.

In addition, the direct adsorption method is a method for measuring the amount of signal derived from an exosome contained in a sample as follows. A sample containing an exosome is brought into contact with a solid phase, without immobilizing the above-described first antibody on the solid phase, so that the exosome is directly adsorbed on the solid phase; the above-described labeled antibody made by modifying the second antibody is added thereto to form a complex; and the label is detected.

### SUMMARY

However, in the sandwich method it is impossible to capture an exosome on a solid phase in a case where the amount of a first protein expressed is low, and the amount of exosome adsorbed is limited by the amount of protein expressed on a membrane surface of the exosome which is recognized by an antibody.

In addition, in the direct adsorption method, in a case where there is a large amount of contaminant protein in a sample, the amount of protein which is non-specifically adsorbed to a solid phase is increased and the amount of exosome adsorbed is limited.

The present invention has been made in consideration of the above-described circumstances, and an object of the present invention is to provide an exosome analysis method, an exosome analysis chip, and an exosome analysis device through which it is possible to analyze an exosome with high sensitivity.

The present inventors have conducted extensive studies in order to solve the above-described problems, and as a result they have found that the problems can be solved by immobilizing an exosome using a substrate which is modified with a compound having a hydrophobic chain and a hydrophilic chain. An embodiment of the present invention provides the following (1) to (3).

(1) An exosome analysis method according to an embodiment of the present invention includes:

(a) a process of bringing an exosome-containing sample into contact with a substrate which is modified with a compound having a hydrophobic chain and a hydrophilic chain to bind the exosome to the compound;

(b) a process of bringing the exosome into contact with a first molecule which specifically binds to a biomolecule existing on the surface of the exosome to form a first molecule-exosome complex on the substrate; and

(c) a process of detecting the first molecule-exosome complex on the substrate.

(2) An exosome analysis chip according to an embodiment of the present invention includes:

an inlet;

a testing unit which has a layer modified with a compound having a hydrophobic chain and a hydrophilic chain; and

a flow path which connects the inlet to the testing unit.

(3) An exosome analysis device according to an embodiment of the present invention includes:

a stage on which an exosome analysis chip provided with a testing unit having a layer modified with a compound having a hydrophobic chain and a hydrophilic chain is placed; and

a detection unit which detects an exosome which is immobilized to the layer of the testing unit by irradiating the testing unit with light.

According to the present invention, it is possible to immobilize a minute amount of exosome in a sample without limiting the amount of exosome adsorbed, and to detect and analyze the exosome with high sensitivity and high accuracy.

### BRIEF DESCRIPTION OF THE DRAWINGS

FIG. 1A is a schematic view of an aspect of the process (c) of an exosome analysis chip in the present embodiment.

FIG. 1B is a schematic view of an aspect of the process (c) of the exosome analysis chip in the present embodiment.

FIG. 2 is a schematic view of an aspect of an exosome analysis chip in the present embodiment.

FIG. 3 is a schematic view of an aspect of an exosome analysis chip in the present embodiment.

FIG. 4 is a schematic view of an aspect of an exosome analysis chip in the present embodiment.

FIG. 5 is a schematic view of an aspect of an exosome analysis chip in the present embodiment.

FIG. 6 is a schematic view of an aspect of an exosome analysis chip in the present embodiment.

FIG. 7 is a schematic view of an aspect of an exosome analysis device in the present embodiment.

FIG. 8A shows a result of a fluorescent observation of exosomes immobilized on a BAM substrate in Example.

FIG. 8B shows a result of a fluorescent observation of exosomes immobilized on a BAM substrate in Example.

FIG. 9 shows a result of quantitative determination of exosomes immobilized to a BAM substrate in Example.

FIG. 10 shows a result of quantitative determination of exosomes immobilized to a BAM substrate in Example.

### DESCRIPTION OF EMBODIMENTS

<<Exosome Analysis Method>>

An exosome analysis method of the present embodiment includes:

(a) a process of bringing an exosome-containing sample into contact with a substrate which is modified with a compound which has a hydrophobic chain and a hydrophilic chain to bind the exosome to the compound which has a hydrophobic chain and a hydrophilic chain on the substrate;

(b) a process of bringing the exosome into contact with a first molecule which is specifically bound with a biomolecule existing on the surface of the exosome to form a first molecule-exosome complex on the substrate; and

(c) a process of detecting the first molecule-exosome complex on the substrate.

The exosome is a secretion of a cell and express on their surface biomolecules, for example, proteins, nucleic acids, sugar chains, and glycolipids, which are derived from the cell of a secretion source on the surface of the exosome. An abnormal cell such as a cancer cell existing within a living body expresses a specific protein in the cell membrane. For this reason, it is possible to detect an abnormality of the cell of the secretion source by analyzing proteins expressed on the surface of the exosome. Here, the surface of the exosome is a surface of a membrane of a membrane vesicle which is secreted from the cell, and refers to a section in which the secreted exosome comes into contact with an environment within a living body.

Furthermore, the exosome is detected in a body fluid, such as blood circulating within a living body, urine, and saliva. Therefore, it is possible to detect an abnormality within a living body by analyzing the exosome without performing a biopsy examination.

Hereinafter, each process will be described.

The process (a) is a process of bringing an exosome-containing sample into contact with a substrate which is modified with a compound which has a hydrophobic chain and a hydrophilic chain to bind the exosome to the compound which has a hydrophobic chain and a hydrophilic chain on the substrate.

The compound which has a hydrophobic chain and a hydrophilic chain is a compound having a hydrophobic chain in order to be bound to a lipid bilayer membrane, and a hydrophilic chain in order to make this lipid chain soluble. By using the compound, it is possible to immobilize an exosome having a lipid bilayer membrane on a substrate.

In the present specification, the term “immobilization of an exosome on a substrate” also includes adsorption of an exosome on a substrate.

The hydrophobic chain may be a single chain or a multiple chain, and examples thereof include a saturated or unsaturated hydrocarbon group which may have a substituent group.

As the saturated or unsaturated hydrocarbon group, a 6C-24C straight-chain or branched-chain alkyl group or alkenyl group is preferable, and examples thereof include a hexyl group, a heptyl group, an octyl group, a nonyl group, a decyl group, an undecyl group, a dodecyl, a tridecyl group, a tetradecyl group, a pentadecyl group, a hexadecyl group, a heptadecyl group, a stearyl group (octadecyl group), a nonadecyl group, an icosyl group, a heneicosyl group, a docosyl group, a tricosyl group, a tetracosyl group, a myristoleyl group, a palmitoleyl group, an oleyl group, a linoyl group, a linoleyl group, a ricinoleyl group, and an isostearyl group.

Among these, a myristoleyl group, a palmitoleyl group, an oleyl group, a linoyl group, and a linoleyl group are preferable, and an oleyl group is more preferable.

Examples of the hydrophilic chain include proteins, oligopeptides, polypeptides, polyacrylamide, polyethylene glycol (PEG), and dextran, and PEG is preferable.

The hydrophilic chain is preferably modified chemically for bonding to a substrate, more preferably has an active ester group, and particularly preferably has an N-hydroxysuccinimide group (NHS group).

That is, as the compound having a hydrophobic chain and a hydrophilic chain, a lipid-PEG derivative is preferable.

The lipid-PEG derivative is called a biocompatible anchor for membrane (BAM). Examples of the BAM include a compound represented by the following Formula (1).

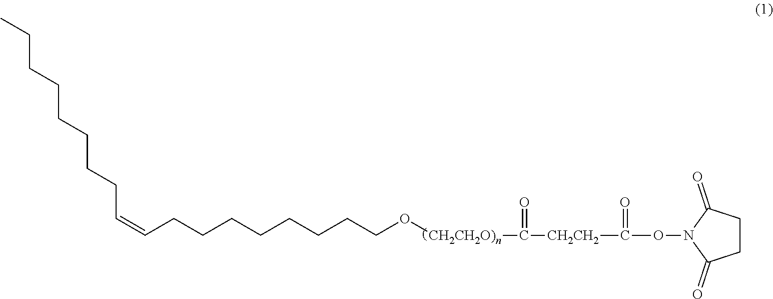

[In the formula, n represents an integer greater than or equal to 1.]

Examples of the substrate used in the process (a) include a glass substrate, a silicon substrate, a polymer substrate, and a metal substrate. The substrate may bind to the compound having a hydrophobic chain and a hydrophilic chain through a substance that binds to the hydrophilic chain of the compound. Examples of the substance include a substance having an amino group, a carboxyl group, a thiol group, a hydroxyl group, and an aldehyde group, and 3-aminopropyltriethoxysilane is preferable.

The exosome-containing sample is not particularly limited as long as the exosome-containing sample is a sample which has been obtained from an environment surrounding a cell to be detected and contains an exosome secreted by the cell, and examples thereof include a sample such as blood, urine, breast milk, bronchoalveolar lavage fluid, amniotic fluid, malignant effusion, or saliva. Among these, blood or urine, from which it is easy to detect an exosome, is preferable. Furthermore, in blood, blood plasma is preferable in view of ease of detecting an exosome.

In addition, the sample also includes a cell culture solution which contains an exosome secreted by a culture cell.

As the exosome-containing sample, an exosome-containing sample prepared through ultracentrifugation, ultrafiltration, continuous flow electrophoresis, filtration using a size filter, or gel filtration chromatography may be used. In addition, in the present embodiment, the exosome-containing sample may be a crude sample which has not been processed.

Examples of the cell to be detected include a cancer cell, a mast cell, a dendritic cell, a reticulocyte, an epithelial cell, a B cell, and a neuron.

The process (a) is preferably a process of specifically binding an exosome to a compound having a hydrophobic chain and a hydrophilic chain on the substrate. Examples of the method for specifically binding an exosome to the substrate include a method for providing a non-specific adsorption suppression portion to a substrate. Examples thereof include a method comprising modifying a substrate with a compound having a hydrophobic chain and a hydrophilic chain, and then treating a site which has not been modified with the compound having a hydrophobic chain and a hydrophilic chain with a compound having a hydrophilic chain such as PEG.

The process (b) is a process of bringing the exosome into contact with a first molecule which is specifically bound to a biomolecule existing on the surface of the exosome to form a first molecule-exosome complex on the substrate.

In the process (b), the contact includes an interaction between, for example, a biomolecule existing on the surface of an exosome and a first molecule which is specifically bound to the biomolecule. Examples of the interaction include a binding reaction such as an antigen-antibody reaction.

An abnormal cell secreting an exosome expresses on its surface a specific protein as a biomolecule, or such abnormal cell is deficient in expressing a specific protein.

Accordingly, it is possible to detect an abnormality in a cell by using an antibody, as a first molecule, which recognizes proteins having a different expression pattern compared to the pattern of a normal cell as an antigen.

From this viewpoint, it is preferable to use an antibody recognizing proteins which are highly expressed in an abnormal cell or a normal cell as an antigen. It is more preferable to use antibody recognizing proteins which are specifically expressed in an abnormal cell or a normal cell as an antigen.

In addition, as the first molecule, an aptamer can also be suitably used without being limited to be an antibody. Examples of the aptamer include a nucleic acid aptamer or a peptide aptamer.

For example, it has been reported that proteins such as CD10, CD5/6, CAV1, MOESIN, or ETS1 are highly expressed in a mammary gland epithelial cell line, whereas the expression of these kinds of proteins is decreased in a breast cancer cell line (Charafe-Jauffret E, et. al., Oncogene (2006) vol. 25, pp. 2273-2284). In a case of detecting an abnormality of a mammary gland epithelial cell, antibodies thereto are used, for example.

In view of ease of forming of a complex with an exosome by an antibody, it is more preferable that a membrane protein such as a receptor be used as an antigen.

Accordingly, in a case of detecting an abnormality of a mammary gland epithetical cell, it is preferable to use an antibody that targets a membrane protein such as CD10, CD5/6, or CD44, as an antigen.

In addition, the first molecule may include different kinds of antibodies or aptamers, or a combination thereof, or may be a molecule which recognizes different epitopes in an identical biomolecule. By using the first molecule, it is possible to improve the accuracy of recognizing an exosome which has a specific biomolecule.

In addition, the different kinds of antibodies or aptamers, or a combination thereof may recognize different biomolecules. For example, by using a plurality of kinds of antibodies recognizing a plurality of kinds of proteins which are highly expressed in a breast cancer cell or a normal mammary gland epithelial cell as antigens, it is possible to improve the accuracy when detecting an abnormality of a mammary gland epithelial cell.

The antibodies or the aptamers to be used are not limited to be related to cancer, and may be related to obesity, diabetes, neurodegenerative disease, or the like. By using the antibodies or the aptamers, it is possible to detect an abnormality relating to a disease in a target cell.

The process (c) is a process of detecting the first molecule-exosome complex on a substrate.

The process (c) is, for example, a process of detecting the first molecule-exosome complex which has been labeled. For example, a labeled molecule which specifically interacts with the first molecule is made to react with the first molecule-exosome complex. Examples of the method for labeling include fluorescent labeling or enzyme labeling. In this manner, it is possible to selectively detect the labeled first molecule-exosome complex.

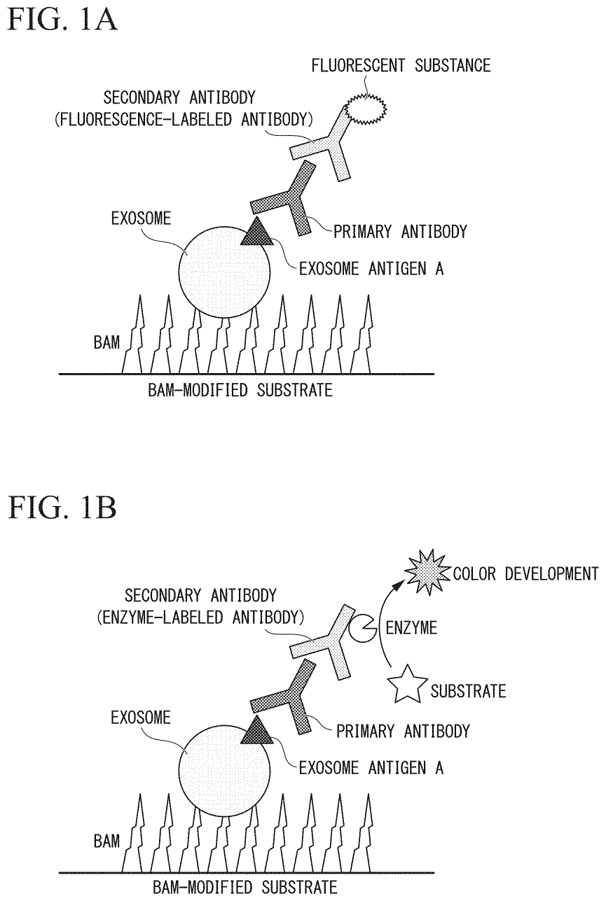

The process (c) is, for example, a process of detecting fluorescence of the first molecule-exosome complex which has been subjected to fluorescent labeling. For example, in a case of using an antibody as a first molecule, it is preferable to use an antibody which is obtained by labeling a secondary antibody to the antibody used in the process (b) with an enzyme such as peroxidase and alkaline phosphatase, or with nanoparticles such as gold colloids or quantum dots (refer to FIG. 1A).

Examples of the quantum dots include CdSe or CdTe. These quantum dots are excellent in that they are bright compared to conventional organic dyes or conventional fluorescent proteins and they are not easily faded by light.

In addition, a detection method using ELISA may be used. Examples thereof include a method comprising reacting the enzymatically labeled secondary antibody with a primary antibody, and detecting color development of the enzyme reaction product by adding a substrate with color-developing properties (refer to FIG. 1B). Even in this case, similarly to the case of the above-described fluorescent labeling, it is possible to detect an exosome using an exosome analysis device to be described below.

In blood, extracellular vesicles such as microvesicles or apoptotic bodies are contained in addition to an exosome, and there is a possibility that these extracellular vesicles will also be immobilized to a substrate. From the viewpoint of removing these extracellular vesicles from the substrate, it is preferable to have a process of washing an exosome on the substrate. The washing process is preferably provided after the process (a) in which an exosome is immobilized to a substrate, after the process (b) in which a first molecule is brought into contact with the exosome to form a first molecule-exosome complex on the substrate, and after the process (c) in which the first molecule-exosome complex is subjected to fluorescent labeling.

In the washing process in the present embodiment, since the binding of the exosome to the substrate which is modified with a compound having a hydrophobic chain and a hydrophilic chain is strong, it is possible to adjust the flow rate to be fast. Thus, the washing can be performed within a short period of time. In addition, the flow rate in the washing process is, for example, less than or equal to 10 mm/s, and an example thereof includes less than or equal to 5 mm/s.

Hereinafter, the exosome analysis method of the present embodiment will be described in detail using an exosome analysis chip of the present embodiment.

### <<Exosome Analysis Chip>>

### First Embodiment

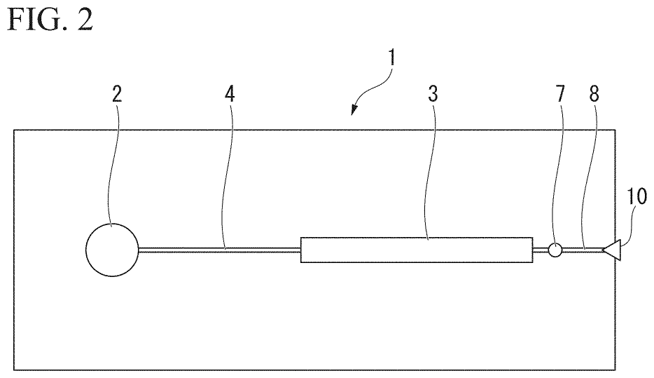

FIG. 2 is a schematic view showing a basic configuration of an exosome analysis chip 1 of the present embodiment.

The exosome analysis chip 1 of the present embodiment includes an inlet 2; a testing unit 3 which has a layer modified with a compound having a hydrophobic chain and a hydrophilic chain; and a flow path 4 which connects the inlet 2 to the testing unit 3.

The exosome analysis chip 1 of the present embodiment further includes, for example, an outlet 10; and a flow path 8 which has a valve 7 and connects the outlet 10 to the testing unit 3. The outlet 10 has a function of discharging a waste liquid. In addition, the outlet 10 also has a function as a connector with a suction pump or the like in a case of performing suctioning, and also has a function as an air ventilator such as a vent filter in a case of pushing and feeding a liquid from an inlet or in a case where there is a driving force within an exosome analysis chip. The valve 7 is appropriately opened and closed in accordance with the washing process or the like.

In addition, the exosome analysis chip 1 of the present embodiment may have a waste liquid tank following the testing unit. In addition, the exosome analysis chip 1 of the present embodiment may have both of an outlet and a waste liquid tank. For example, the exosome analysis chip includes an outlet following the waste liquid tank.

Examples of an exosome analysis method using the exosome analysis chip 1 of the present embodiment include the following method.

First, an exosome-containing sample which has been prepared from a blood specimen is injected into the inlet 2. The exosome-containing sample which has been prepared from the blood specimen and is injected into the inlet 2 reaches the testing unit 3 through the flow path 4. The testing unit 3 has a layer modified with a compound having a hydrophobic chain and a hydrophilic chain, and therefore, the hydrophobic chain on this layer captures an exosome having a lipid bilayer membrane.

In the following embodiment, the blood specimen may be directly injected into the inlet 2 instead of the exosome-containing sample prepared from the blood specimen.

Next, an antibody is injected as a first molecule into the inlet 2. The antibody reaches the testing unit 3 through the flow path 4. In a case where there is a protein as a biomolecule recognized by the injected antibody, on the surface of an exosome immobilized to the testing unit 3, an antibody-exosome complex is formed on the testing unit 3.

Next, a secondary antibody which has been subjected to fluorescent labeling is injected into the inlet 2. In a case where the antibody-exosome complex is formed on the testing unit 3, the injected secondary antibody is bound to this complex to further form a complex. In this case, the testing unit 3 emits fluorescence due to the fluorescence with which the secondary antibody is labeled.

According to the present embodiment, it is possible to analyze whether or not an exosome-containing sample expresses a predetermined biomolecule on the surface thereof. As described above, the surface of a membrane of an exosome expresses proteins derived from a cell of a secretion source, and therefore, it is possible to detect an abnormality of the cell of a secretion source through analysis of the exosome.

In addition, the affinity between the exosome and the compound having a hydrophobic chain and a hydrophilic chain with which the top of a substrate is modified is high, and therefore, the exosome in the sample injected into the inlet 2 is immediately immobilized to the testing unit 3. Therefore, according to the present embodiment, the time for adsorption becomes short, and thus, it is possible to analyze the exosome within a short period of time.

### Second Embodiment

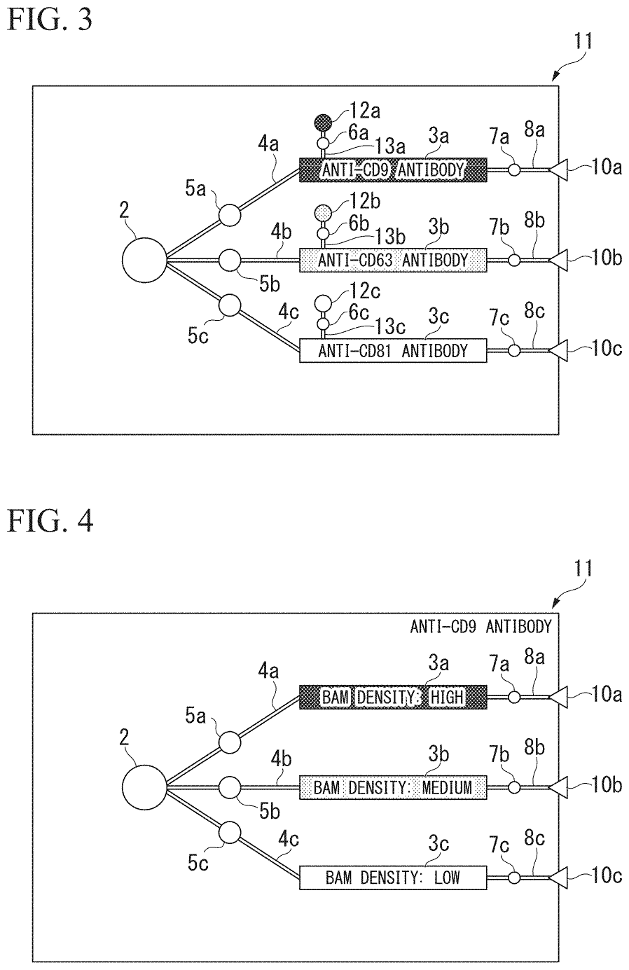

FIG. 3 is a schematic view showing a basic configuration of an exosome analysis chip 11 of the present embodiment.

The exosome analysis chip 11 of the present embodiment includes an inlet 2; a plurality of testing units 3a, 3b, and 3c having a layer modified with a compound having a hydrophobic chain and a hydrophilic chain; and flow paths 4a, 4b, and 4c which respectively have valves 5a, 5b, and 5c and connect the inlet 2 to the testing units 3a, 3b, and 3c.

The exosome analysis chip 11 of the present embodiment further includes, for example, outlets 10a, 10b, and 10c; and flow paths 8a, 8b, and 8c which respectively have valves 7a, 7b, and 7c and connect the outlets 10a, 10b, and 10c to the testing units 3a, 3b, and 3c.

The testing units 3a, 3b, and 3c are respectively connected to, for example, inlets 12a, 12b, and 12c through flow paths 13a, 13b, and 13c which respectively have valves 6a, 6b, and 6c.

In addition, the exosome analysis chip 11 of the present embodiment may have waste liquid tanks following the testing units. In addition, the exosome analysis chip 11 of the present embodiment may have both of outlets and waste liquid tanks. For example, the exosome analysis chip includes outlets following the waste liquid tanks.

Examples of an exosome analysis method using the exosome analysis chip 11 of the present embodiment include a method for bringing different kinds of antibodies or aptamers into contact with exosomes on the respective testing units to form antibody- or aptamer-exosome complexes, in the process (b).

An example thereof includes the following method.

First, an exosome-containing sample which has been prepared from a blood specimen is injected into the inlet 2. At this time, the valves 5a, 5b, and 5c enter an open state and valves 6a, 6b, and 6c enter a closed state.

The exosome-containing sample which has been prepared from the blood specimen and is injected into the inlet 2 reaches the testing units 3a, 3b, and 3c through the flow paths 4a, 4b, and 4c. Exosomes are captured by layers modified with a compound having a hydrophobic chain and a hydrophilic chain of the testing units 3a, 3b, and 3c.

Next, the valves 5a, 5b, and 5c are made to enter a closed state and the valve 6a is made to enter an open state. An anti-CD9 antibody is injected as a first molecule into the inlet 12a. The anti-CD9 antibody reaches the testing unit 3a through the flow path 13a. In a case where there is CD9 on the surface of the exosome immobilized to the testing unit 3a, an anti-CD9 antibody-exosome complex is formed on the testing unit 3a.

Next, the valve 6b is made to enter an open state and an anti-CD63 antibody is injected as a second molecule into the inlet 12b. In a case where there is CD63 on the surface of the exosome immobilized to the testing unit 3b, an anti-CD63 antibody-exosome complex is formed on the testing unit 3b.

Next, the valve 6c is made to enter an open state and an anti-CD81 antibody is injected as a third molecule into the inlet 12c. In a case where there is CD81 on the surface of the exosome immobilized to the testing unit 3c, an anti-CD81 antibody-exosome complex is formed on the testing unit 3c.

Next, the valves 5a, 5b, and 5c are made to enter an open state and the valves 6a, 6b, and 6c are made to enter a closed state. A secondary antibody which has been subjected to fluorescent labeling is injected into the inlet 2. In a case where the antibody-exosome complexes are formed on the testing units 3a, 3b, and 3c, the injected secondary antibody is bound to these complexes to further form complexes. In this case, the testing units 3a, 3b, and 3c emit fluorescence due to the fluorescence with which the secondary antibody is labeled.

According to the present embodiment, it is possible to analyze whether or not an exosome-containing sample expresses a plurality of predetermined biomolecules on the surface thereof, at one time.

By using a plurality of molecules which are specifically bound to biomolecules existing on the surface of an exosome, it is possible to specify characteristics different from those of a detected cell specified using a first molecule. For example, in a case where a molecule which recognizes proteins specifically expressed in cancer as an antigen is used as a first molecule, a molecule which recognizes proteins specifically expressed in a certain organ as an antigen is used as a second molecule. Accordingly, it is possible not only to specify whether or not a cell of a secretion source of an exosome is a cancer cell, but also to specify which internal organ in a living body the cell of the secretion source of the exosome is derived from.

Examples of proteins which are specifically expressed in a certain organ include a prostate cancer marker such as PSA, PSCA, or PSMA; and a breast cancer marker such as CA15-3, BCA225, or HER2. By using antibodies, as second molecules, which recognize these as antigens, it is possible to specify type of cancer of the detected cancer cell.

In addition, as shown in FIG. 4, one kind of antibody (for example, anti-CD9 antibody) may be used as a first molecule, and the densities (for example, BAM densities) of compounds having a hydrophobic chain and a hydrophilic chain may be different from each other in each testing unit. Accordingly, it is possible to analyze expression of a predetermined protein in an exosome-containing sample under optimum conditions without saturation of the amount of exosome captured by the testing units.

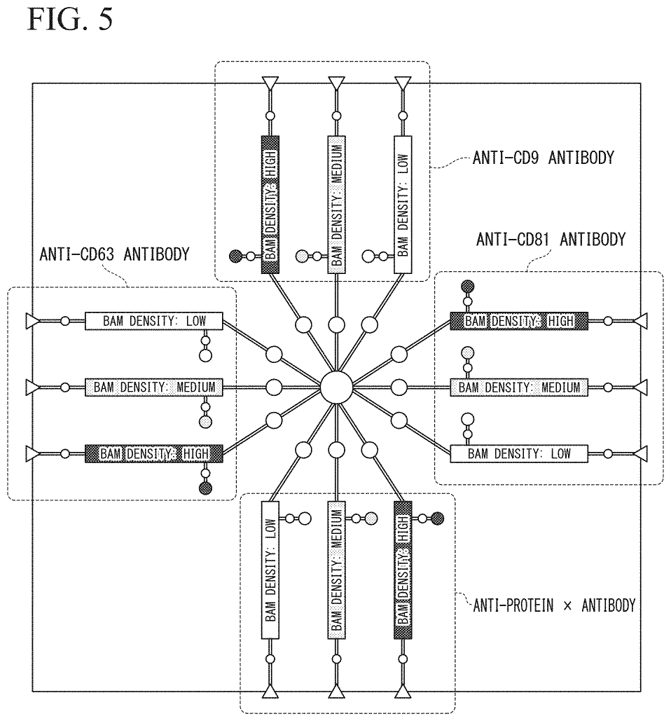

Furthermore, as shown in FIG. 5, plural kinds of molecules (for example, an anti-CD9 antibody, an anti-CD63 antibody, an anti-CD81 antibody, and an anti-protein X (arbitrary protein) antibody) may be used, and the densities (for example, BAM densities) of compounds having a hydrophobic chain and a hydrophilic chain may be different from each other in each testing unit. Accordingly, it is possible to analyze expression of a plurality of a predetermined protein in an exosome-containing sample under optimum conditions at one time without saturation of the amount of exosome captured by the testing units.

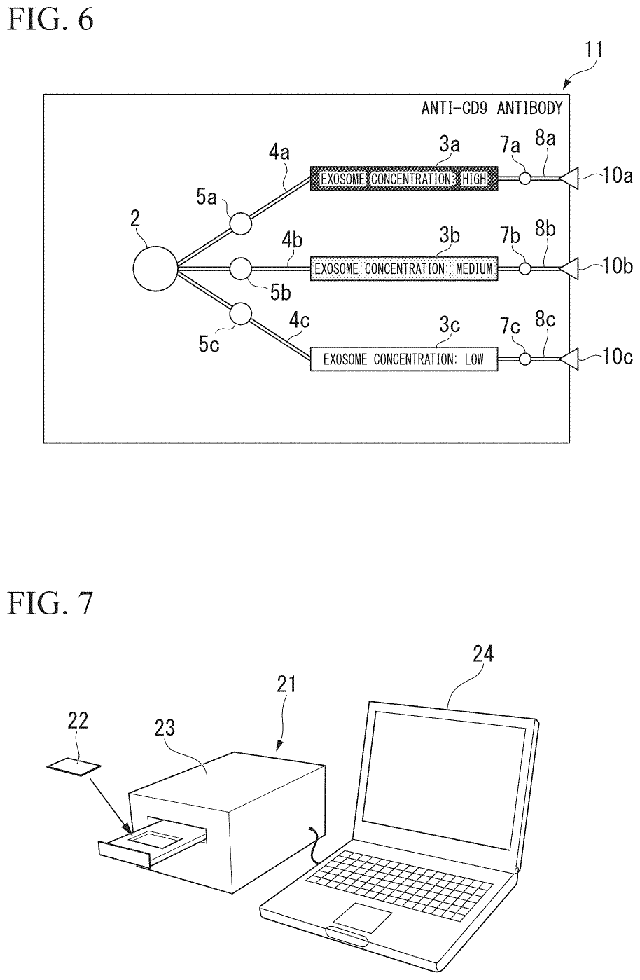

In addition, as shown in FIG. 6, in the process (a), exosome-containing samples having different concentrations from each other may be brought into contact with layers modified with compounds having a hydrophobic chain and a hydrophilic chain on each testing unit to immobilize exosomes to the testing units. Accordingly, it is possible to analyze expression of a predetermined protein in exosome-containing samples under optimum conditions without saturation of the amount of exosome captured by the testing units.

### <<Exosome Analysis Device>>

An exosome analysis device of the present embodiment has a detection unit which detects the exosomes adsorbed on the testing units of the above-described exosome analysis chip.

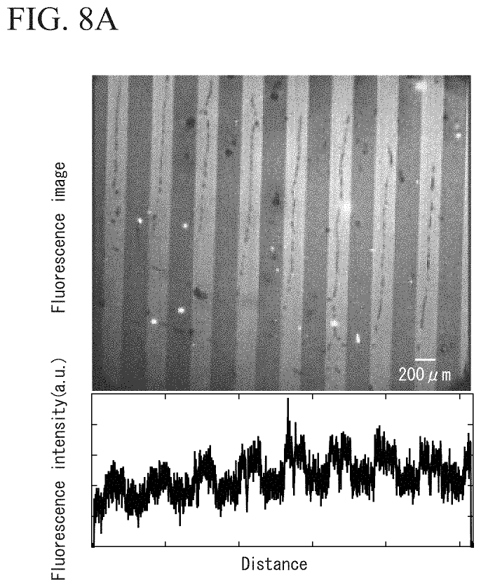

An embodiment of the exosome analysis device of the present embodiment will be described with reference to FIG. 7.

As shown in FIG. 7, an exosome analysis device 21 of the present embodiment includes a detection unit which detects analysis results. The exosome analysis device 21 has, for example, a light source (not shown), an exosome analysis chip 22, a detection unit 23, and a control unit 24 such as a personal computer.

As the light source, it is possible to use, for example, a light source (for example, an ultraviolet lamp or a visible light lamp) which can emit light such as ultraviolet rays or visible light.

Analysis results of an exosome immobilized to a testing unit of the exosome analysis chip 22 are detected through the detection unit 23.

Hereinafter, the present invention will be described using Example, but the present invention is not limited to the following Example.

### EXAMPLE

### [Immobilization of Exosome on BAM Substrate]

A glass substrate was modified with 3-aminopropyltriethoxysilane (hereinafter, also referred to as APTES), an amino group of APTES was reacted with an NHS group of BAM represented by the above Formula (1), and the substrate was modified with an oleyl group which selectively immobilizes a lipid bilayer membrane. Furthermore, NHS-PEG-OCH₃ was reacted in order to suppress non-specific adsorption. Thereafter, a BAM substrate was obtained by irradiating vacuum ultraviolet rays through a mask and patterning the modified layer.

Exosomes were collected from human serum through ultracentrifugation. Exosome-containing solutions having various concentrations (2.0×10⁷ particles/mL, 2.0×10⁸ particles/mL, 2.0×10⁹ particles/mL, and 2.0×10¹⁰ particles/mL) were prepared and were reacted with the BAM substrate at RT for 30 mins.

Next, blocking was performed at RT for 1 hr using a skim milk solution (1% skim milk, 0.1% Tween 20 in PBS).

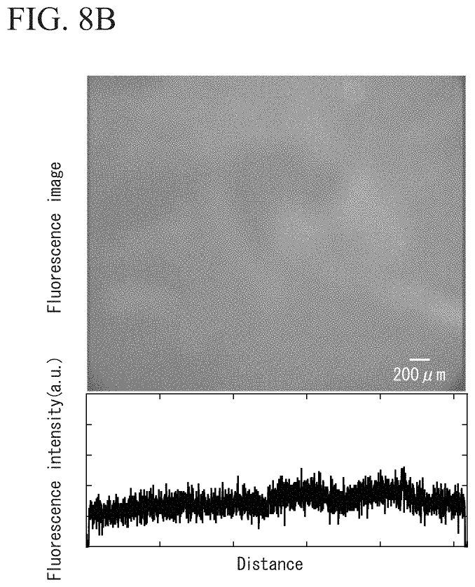

Next, a reaction was performed at RT for 30 mins using anti-human CD9 antibodies (mouse; 2 μg/mL in PBS), and then quantum dot-modified anti-mouse IgG antibodies were reacted to perform fluorescent observation. The observation results are shown in FIG. 8.

As shown in FIG. 8A, a high luminance was observed on the patterned BAM substrate.

It is considered that this is because exosomes are immobilized on the BAM substrate, and quantum dots are specifically bound to the exosomes through antibodies.

In contrast, as shown in FIG. 8B, no high luminance was observed in negative control using a solution containing no exosome. This shows that no antibody is non-specifically adsorbed on the BAM substrate to which no exosome is immobilized.

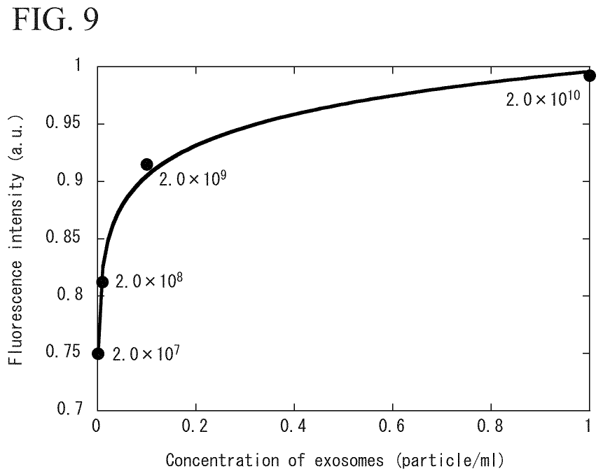

In addition, it was observed that the fluorescence intensity increased as the concentration of exosomes in the solution used became higher, and the fluorescence intensity was gradually saturated as shown in FIG. 9. This result suggests that it is possible to quantitatively determine the amount of immobilized exosome from the fluorescence intensity by previously preparing a calibration curve in which the correlation between the absolute amount of immobilized exosome and the fluorescence intensity is used.

[Relationship Between Number Density of Immobilized Exosomes and Distance from Introduction Unit on Substrate]

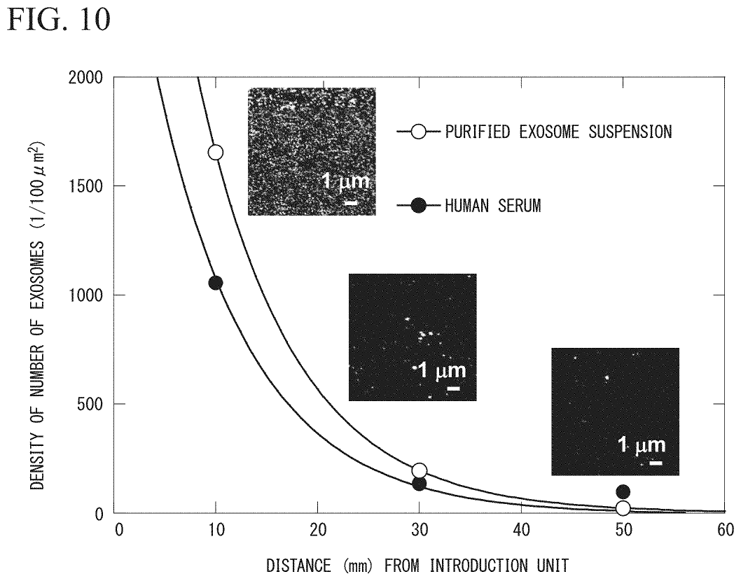

A substrate was fixed in a device, and 1 mL of a sample was introduced at a flow rate of 16.2 mL/min. The number of particles with diameters of 30 nm to 200 nm on the surface of the substrate was counted through an AFM method in a solution, and the number density of exosomes was measured. Human serum and a purified exosome suspension, which was obtained by suspending exosomes collected from human breast cancer MCF-7 cell culture supernatant through a differential ultracentrifugation method, in physiological saline, were respectively used as samples. The results representing the relationship between the number density of immobilized exosomes and the distance from the introduction unit on the substrate are shown in FIG. 10. As shown in FIG. 10, it was confirmed that it is possible to immobilize exosomes to the same extent in human serum and the purified solution derived from the culture supernatant.

From the above results, according to the present embodiment, by using a substrate modified with a compound having a hydrophobic chain and a hydrophilic chain, it is possible to immobilize a minute amount of exosomes in a sample and to analyze the exosomes with high sensitivity.

### REFERENCE SIGNS LIST

1, 11, 22 . . . exosome analysis chip, 2, 12a, 12b, 12c . . . inlet, 3, 3a, 3b, 3c . . . testing unit, 4, 4a, 4b, 4c, 8a, 8b, 8c, 13a, 13b, 13c . . . flow path, 5a, 5b, 5c, 7, 7a, 7b, 7c . . . valve, 10, 10a, 10b, 10c . . . outlet, 21 . . . exosome analysis device, 23 . . . detection unit, 24 . . . control unit

What is claimed is:

1. An exosome analysis method, comprising:
(a) bringing an exosome-containing sample into contact with a substrate which is modified with a compound having a hydrophobic chain and a hydrophilic chain to bind the exosome to the compound;
(b) bringing the exosome into contact with a first molecule that specifically binds to a biomolecule existing on the surface of the exosome to form a first molecule-exosome complex on the substrate; and
(c) detecting the first molecule-exosome complex on the substrate.

2. The exosome analysis method according to claim 1,
wherein the hydrophobic chain contains lipid.

3. The exosome analysis method according to claim 1,
wherein the compound contains a lipid-PEG derivative.

4. The exosome analysis method according to claim 1,
wherein the substrate has a nonspecific adsorption suppression portion.

5. The exosome analysis method according to claim 1,
wherein the first molecule is an antibody, an aptamer, or a combination thereof.

6. The exosome analysis method according to claim 1,
wherein the process (c) comprises quantitatively determining a label of the first molecule-exosome complex that has been labeled.

7. The exosome analysis method according to claim 6,
wherein the quantitative determination of the label is performed using a previously obtained calibration curve showing a relationship between the concentration of exosomes and the amount of the label.

8. The exosome analysis method according to claim 6,
wherein the label is bonded to the first molecule or a second molecule that specifically binds to the first molecule.

9. The exosome analysis method according to claim 1, further comprising:
washing the surface of the substrate.

10. The exosome analysis method according to claim 1,
wherein, as the substrate, a substrate that is provided with an inlet, a testing unit having a layer modified with the compound having a hydrophobic chain and a hydrophilic chain, and a flow path connecting the inlet to the testing unit is used.

11. The exosome analysis method according to claim 10,
wherein the process (a) comprises introducing the exosome-containing sample from the inlet at a constant speed and binding the exosome with the compound of the testing unit; and
wherein the process (b) comprises introducing a sample that contains the first molecule from the inlet, to form a first molecule-exosome complex in the testing unit.

12. The exosome analysis method according to claim 10,
wherein the substrate includes two or more of the testing units, and each of the testing unit has a layer modified with compounds having a hydrophobic chain and a hydrophilic chain, and each of the layer has a different density of the compounds.

13. The exosome analysis method according to claim 10,
wherein the substrate includes two or more of the testing units, all of which have an identical layer modified with a compound having a hydrophobic chain and a hydrophilic chain.

14. The exosome analysis method according to claim 13,
wherein the process (b) comprises introducing different kinds of first molecules for each testing unit, to form a first molecule-exosome complex in each of the testing units.

15. The exosome analysis method according to claim 10,
wherein the flow path has a valve.
